(19) United States
(12) Patent Application Publication
(10) Pub. No.: US 2015/0265559 A1
(43) Pub. Date: Sep. 24, 2015

(54) BENZOATES FOR USE IN TREATING DEMENTIA

(71) Applicants: Kaohsiung Chang Gung Memorial Hospital of the C.G.M.F., Kaohsiung City (TW); China Medical University, Taichung (TW)

(72) Inventors: Hsien-Yuan LANE, Taichung (TW); Chieh-Hsin Lin, Kaohsiung (TW)

(21) Appl. No.: 14/637,347

(22) Filed: Mar. 3, 2015

Related U.S. Application Data
(60) Provisional application No. 61/955,521, filed on Mar. 19, 2014.

(51) Int. Cl. A61K 31/192; A61K 45/06

(52) U.S. Cl. CPC A61K 31/192; A61K 45/06

(57) ABSTRACT

A method for treating a subject afflicted with dementia is disclosed. The method comprises administering to the subject a composition comprising an effective amount of a benzoic acid, a salt, an ester, or a derivative thereof, and a pharmaceutically acceptable carrier or vehicle, wherein the subject is not administered any other neuropharmaceutical.

### REFERENCE TO RELATED APPLICATION

The present application claims the priority to U.S. Provisional Application Serial No. 61/955,521, filed Mar. 19, 2014, which is herein incorporated by reference in its entirety.

### FIELD OF THE INVENTION

The present invention relates to treatment of dementia.

### BACKGROUND OF THE INVENTION

The prevalence of dementia in elderly persons is increasing rapidly in the aging society, of which the deteriorating clinical course is a heavy burden to both the patients and their family. Early detection and intervention of Alzheimer disease (AD) is pivotal for the outcome. Mild cognitive impairment (MCI), particularly amnestic mild cognitive impairment (aMCI), is a risk factor and might he a prodromal stage of AD. The main-stream treatment for mild and moderate AD is acetylcholine esterase inhibitor (AChEI). However, its efficacy and tolerability are unsatisfactory. Furthermore, AChEI does not show convincing efficacy for MCI, implying that other mechanism(s) might underlie the pathogenesis of early-phase AD.

Excessive glutamatergic neurotransmission, particularly through the N-methyl-D-aspartate receptor (NMDAR), leads to neurotoxicity, which is implicated in the pathophysiology of AD, especially in the late phase. The NMDAR antagonists are developed for the treatment of AD, on the basis of the hypothesis of NMDAR overactivation. Memantine is an uncompetitive NMDAR partial antagonist of low affinity, which supposedly can block NMDAR overactivation by preventing excessive influx of calcium. Memantine is approved as an antidementia medication for mod-erate-severe AD; however, it has limited efficacy at the early phase, including MCI and mild AD. The NMDAR antagonists such as MK-801 also induce apoptosis and neurodegeneration in both in vitro and in vivo studies. Ketamine, another NMDAR antagonist, impaired spatial learning and verbal information ability in healthy humans in a double-blind, randomized, placebo-controlled trial. These findings raise concern that NMDA antagonist might impair cognition and memory in early AD.

Conversely, optimal NMDAR activation is pivotal for synaptic plasticity, memory, and cognitive function. Attenuation of NMDAR-mediated neurotransmission can result in loss of neuronal plasticity and cognitive deficits in the aging brain, which might account for clinical deterioration and brain atrophy. Age-related decrease in the density of NMDAR in cerebral cortex and hippocampus was observed in humans. Earlier studies also found a decrease of glycine-dependent radioligand binding to the NMDAR in cerebral cortices from postmortem and neurosurgical tissues in patients with AD. D-cycloserine, a partial agonist at the glycine site of NMDAR, was reported in some clinical studies to activate the NMDAR in brains of AD patients and improve their score on the cognitive subscale of the Alzheimer's Disease Assessment Scale (ADAS-cog). Recently, 1000 mg/day of sodium benzoate, an inhibitor of D-amino acids oxidase (DAAO), was also found to be beneficial for neurocognitive function in patients with schizophrenia. Because of the supporting, evidence, we propose that NMDA-enhancing agents might be beneficial for the early declining process of AD, due to their role in learning and memory as well as neurogenesis and neuroplasticity.

There are several avenues to enhance NMDA activation. One of them is inhibiting the activity of DAAO, a flavoenzyme of peroxisomes responsible for degrading D-serine and D-alanine, and thereby raising levels of the D-amino acids that are the neurotransmitters for the coagonist site of the NM DAR. Recent data indicate that aging is related to reduced D-serine levels and thereby impaired NMDAR transmission, and D-serine treatment significantly decreases the extent of neuron death, suggesting that D-serine has neuroprotective effect against apoptosis. In addition, neural stem cells from postnatal mouse forebrain can synthesize D-serine and thereby stimulate proliferation and neuronal differentiation of the stem cells.

Enhancing NMDAR through DAAO inhibition might be a safe way to reduce nephrotoxicity of D-serine, particularly in the elderly population. Sodium benzoate is a DAAO inhibitor. Benzoic acid exists in many plants and is a natural constituent of food, including milk products. Benzoic acid and its salts, including sodium benzoate, which are generally recognized as safe, are also food preservatives widely used in manufacturing fruit jelly, buffer, soy-bean sauce, processed meat, and the like.

There are several other preclinical studies supporting, the central nervous system (CNS) effects of DAAO inhibitors, although the memory effect was not examined. Sodium benzoate is effective in NMDAR models such as pain relief and partially prevented cell death in glial cells. The CNS bioavail-ability of benzoate is good. To test the hypothesis that DAAO inhibition is beneficial for the early phase of dementia, we conducted this trial to examine the efficacy and safety of sodium benzoate in patients with aMCI or mild AD.

### SUMMARY OF THE INVENTION

In one aspect, the invention relates to a method of treating dementia, comprising administering to a subject in need of treating the dementia a composition comprising an effective amount of benzoic acid, a salt, an ester or a derivative thereof; and a pharmaceutically acceptable carrier or vehicle, wherein the subject is optionally concurrently administered an acetylcholine esterase inhibitor but not administered any other neuropharmaceutical.

In one embodiment of the invention, the administering step is performed daily for a period selected from the group consisting of more than 6 weeks, at least 8 weeks, at least 16 weeks, at least 20 weeks, and at least 24 weeks.

In another embodiment of the invention, the salt thereof is sodium benzoate.

In another embodiment of the invention, the amount of sodium benzoate administered is at least 250 mg per daily dosage unit. The amount of sodium benzoate administered may be at least 500 mg per daily dosage unit. Alternatively, the amount of the benzoic acid, salt, ester, or derivative thereof administered may be at least 200-2000 mg per daily dosage unit.

In another embodiment of the invention, the salt thereof is sodium benzoate and the amount administered is at least 250-750 mg per daily dosage unit.

In another embodiment of the invention, the administering step comprises; (a) administering to the subject sodium benzoate about 200-500 mg per daily dosage unit for the first treatment period; (b) administering to the subject sodium benzoate about 400-1000 per daily dosage unit for the second treatment period; and (c) administering to the subject sodium benzoate about 500-1000 per daily dosage unit for the second treatment period.

In another embodiment of the invention, the subject is afflicted with Alzheimer disease and/or mild cognitive impairment. The subject may be afflicted with an early phase Alzheimer disease and/or amnestic mild cognitive impairment.

The above method may further comprise measuring and assessing the cognitive function of the subject. The measuring step may be performed by using an Alzheimer's Disease Assessment Scale-cognitive subscale instrument. The measuring step may measure the scores of speed of processing, working memory, and verbal learning, and/or memory tests. The measuring step may measure Clinician's Interview-Based Impression of Change plus Caregiver input.

In another embodiment of the invention, the subject is not concurrently administered an acetylcholine esterase inhibitor or any other neuropharmaceutical.

Further in another aspect, the invention relates to a method of treating dementia, comprising administering to a human subject in need of treating the dementia a composition consisting essentially of benzoic acid, a salt, an ester, or a derivative thereof in an amount effective for inhibition of D-amino acid oxidase in the brain of the subject; and a pharmaceutically acceptable carrier or vehicle, wherein the subject is optionally concurrently administered an acetylcholine esterase inhibitor but not any other neuropharmaceutical.

In one embodiment of the invention, the human subject is not Concurrently administered any acetylcholine esterase inhibitor or any other antidementia compound.

In another aspect, the invention relates to a method of treating dementia in a human subject, comprising administering to the human subject a composition comprising, an effective amount of a D-amino acid oxidase inhibitor; and a pharmaceutically acceptable carrier or vehicle, wherein the subject is not administered any other neuropharmaceutical.

In one embodiment, the D-amino acid oxidase inhibitor is at least one selected from the group consisting of benzoic acid, a salt, an ester, and a derivative thereof. The D-amino acid oxidase inhibitor may be sodium benzoate.

These and other aspects will become apparent from the following description of the preferred embodiment taken in conjunction with the following drawings, although variations and modifications therein may be affected without departing from the spirit and scope of the novel concepts of the disclosure.

The accompanying drawings illustrate one or more embodiments of the invention and, together with the written description, serve to explain the principles of the invention. Wherever possible, the same reference numbers are used throughout the drawings to refer to the same or like elements of an embodiment.

### BRIEF DESCRIPTION OF THE DRAWINGS

FIG. 1 shows a flow diagram and disposition of two treatment groups.

FIG. 2 is a table showing baseline demographic characteristics of placebo and sodium benzoate treatment groups.

FIG. 3 is a table showing Mean ± SD scores of primary and secondary outcomes.

FIG. 4 is a table showing the results of measuring ADAS-cog over 24-week treatment with GEE method in subgroups.

FIG. 5 is a table showing the results of measuring additional cognition composite over 24-week treatment using independent t test in subgroups.

FIG. 6 is a table showing the results of clinical measurement of CIBIC-plus over 24-week treatment using Mann-Whitney U Test in Subgroups.

FIG. 7 is a table showing the results of measuring ADAS-cog over 24-week treatment using generalized estimating equations (GEE) method adjusted for the baseline effect.

### DETAILED DESCRIPTION OF THE INVENTION

The present invention is more particularly described in the following examples that are intended as illustrative only since numerous modifications and variations therein will be apparent to those skilled in the art. Various embodiments of the invention are now described in detail. Referring to the drawings, like numbers indicate like components throughout the views. As used in the description herein and throughout the claims that follow, the meaning of “a”, “an”, and “the” includes plural reference unless the context clearly dictates otherwise. Also, as used in the description herein and throughout the claims that follow, the meaning of “in” includes “in” and “on” unless the context clearly dictates otherwise. Moreover, titles or subtitles may be used in the specification for the convenience of a reader, which shall have no influence on the scope of the present invention. Additionally, some terms used in this specification are more specifically defined below.

### DEFINITIONS

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention. For convenience, certain terms may be highlighted, for example using italics and/or quotation marks. The use of highlighting has no influence on the scope and meaning of a term; the scope and meaning of a term is the same, in the same context, whether or not it is highlighted. It will be appreciated that same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and in no way limits the scope and meaning of the invention or of any exemplified term. Likewise, the invention is not limited to various embodiments given in this specification.

Unless otherwise defined, all technical and scientific terms used herein have the same meaning as commonly understood by one of ordinary skill in the art to which this invention pertains. In the case of conflict, the present document, including definitions will control.

As used herein, “around”, “about” or “approximately” shall generally mean within 20 percent, preferably within 10 percent, and more preferably within 5 percent of a given value or range. Numerical quantities given herein are approximate, meaning that the term “around”, “about” or “approximately” can be inferred if not expressly stated.

As used herein, when a number or a range is recited, ordinary skill in the art understand it intends to encompass an appropriate, reasonable range for the particular field related to the invention.

By at least 200-2000 mg it meant that all integer unit amounts within the range are specifically disclosed as part of the invention. Thus, 200, 201, 202 . . . 250, 251, 252 . . . 1000, 1001, 1002 . . . 1997, 1998, 1999 and 2000 unit amounts are included as embodiments of this invention.

The term “treating” or “treatment” refers to administration of an effective amount of a therapeutic agent to a subject in need thereof with the purpose of cure, alleviate, relieve, remedy, ameliorate, or prevent the disease, the symptoms of it, or the predisposition towards it. Such a subject can be identified by a health care professional based on results from any suitable diagnostic method.

“An effective amount” refers to the amount of an active agent that is required to confer a therapeutic effect on the treated subject. Effective doses will vary, as recognized by those skilled in the art, depending on routes of administration, excipient usage, and the possibility of co-usage with other therapeutic treatment.

As used herein, the term “neuropsychiatric disorder” refers to a disease having a pathophysiological component of attenuated NMDA receptor-mediated neurotransmission. Examples of such disorders include schizophrenia, bipolar disorder, Alzheimer's disease, dementia, autism Asperger's disorder, depression, benign forgetfulness, mild cognitive impairment, childhood learning disorders, close head injury, ataxia, spinocerebellar degeneration, Parkinson's disease, general anxiety disorder, panic disorder, obsessive compulsive disorder, phobia including, social phobia, substance abuse, substance dependence, and attention deficit disorder.

A “neuropharmaceutical” refers to a drug used to treat neuropsychiatric neuropsychological, or nervous-system disorders including, but not limited to depression, schizophrenial, bipolar disorder, attention deficit hyperactivity disorder (AMID), schizophrenia, Alzheimer's disease, and the like. For example, an antipsychotic drug (e.g., risperidone, olanzapine, etc.), and antidepressant (e.g., sertraline, fluoxetine hydrochloride, etc.), a psychotropic, medication for attention deficit and hyperactivity disorder (e.g., Ritalin, Dexedrine, Atomoxetine, etc.), a psychotropic medication for dementia (e.g., Aricept, memantine), and the like). A “neuropharmaceutical” includes, but not limited to, a NMDA-enhancing agent, such as sarcosine.

In certain embodiments the benzoic acid, benzoic acid salt, or derivative thereof is selected from the group consisting of benzoic acid, sodium benzoate, potassium benzoate, calcium benzoate, 2-aminobenzoate, 3-aminobenzoate, and 4-aminobenzoate.

### EXAMPLES

Without intent, to limit the scope of the invention, exemplary instruments, apparatus, methods and their related results according to the embodiments of the present invention are given below. Note that titles or subtitles may be used in the examples for convenience of a reader, which in no way should limit the scope of the invention. Moreover, certain theories are proposed and disclosed herein; however, in no way they, whether they are right or wrong, should limit the scope of the invention so long as the invention is practiced according to the invention without regard for any particular theory or scheme of action.

### Methods and Materials

### Participants

Patients were recruited from outpatient clinics at the Department of Psychiatry and Department of Neurology, Kaohsiung Chang Gung Memorial Hospital, Kaohsiung; Department of Psychiatry, China Medical University Hospital, Taichung; Department of Psychiatry, Taichung Veterans General Hospital, Taichung; and Department of Neurology, Lin-Shin Hospital, Taichung, which are four major medical centers in Taiwan. The study was approved by the institutional review board at four sites and conducted in accordance with the current revision of the Declaration of Helsinki. Patients were evaluated by research psychiatrists and neurologists after a thorough medical and neurological workup.

Patients were enrolled into this study if they: 1) satisfied National Institute of Neurological and Communicative Disorders and Stroke and the Alzheimer's Disease and Related Disorders Association criteria for probable AD and had a Clinical Dementia Rating (CDR) score of 1 or criteria for an aMCI of a presumably degenerative nature defined as subjective memory complaint corroborated by an informant and insufficient global cognitive and functional impairment to meet National Institute of Neurological and Communicative Disorders and Stroke and the Alzheimer's Disease and Related Disorders Association criteria and had a CDR score of .5; 2) 50-90 years of age; 3) were physically healthy and had all laboratory assessments (including urine/blood routine, biochemical tests, and electrocardio-graph) within normal limits; 4) had a Mini-Mental State Examination score of 17-26; 5) had sufficient education to communicate effectively and were capable of completing the assessments of the study; and 6) agreed to participate in the study and provided informed consent. For patients who had already been on a regimen of AChEI therapy, AChEI had to be continued for at least 3 months before enrollment. The AChEI dose had to be kept unchanged during the study duration. For patients who had not yet been on a regimen of AChEI therapy, AChEI or other antidementia medication was forbidden during the study duration.

Exclusion criteria included history of significant cerebrovascular disease; Hachinski Ischemic Score >4; major neurological, psychiatric, or medical conditions other than AD; substance (including alcohol) abuse or dependence; delusion, hallucination, or delirium symptoms; severe visual or hearing loss; and inability to follow protocol.

### Study Design

All patients were randomly assigned to receive a 24-week treatment of sodium benzoate or placebo in a double-blind manner. Efficacy and safety were evaluated at baseline and at the ends of weeks 8, 16, and 24. Two hundred fifty milligrams of sodium benzoate or placebo were packed with identical capsules provided in coded containers. The dose was started at 250-500 mg/day (250 mg once or twice daily) in the first 8 weeks, then increased by 250-500 mg/day from the 9th week, and further increased by another 250-500 mg/day from the 17th week of the study if clinically indicated. On the basis of an earlier study in which sodium benzoate given at the dose of 1000 mg/day improved a variety of symptom domains and neurocognition in patients with chronic schizophrenia without obvious side effects, we decided to apply 250-750 mg/day, considering the older age of the subjects in the present study. Patients were randomized in a cluster of six subjects to receive sodium benzoate or placebo in a 1:1 ratio by an independent investigational pharmacist.

Patients, caregivers, and investigators, except the investigational pharmacist, were all blind to the assignment. Patient medical adherence and safety were closely monitored by care-givers and research physicians, and pill-counting was monitored by the study staff.

### Assessments

The primary outcome was the Alzheimer's Disease Assessment Scale-cognitive subscale (ADAS-cog) measured at weeks 0, 8, 16, and 24. The ADAS-cog is the most popular cognitive assessment instrument used in AD clinical trials. It consists of 11 tasks, including word recall, naming, commands, constructional praxis, ideational praxis, orientation, word recognition, instructions remembering, spoken language ability, word-finding difficulty, and comprehension. Its scores range from 0 (best) to 70 (worst).

The secondary outcome measurements included the Clinician's Interview-Based Impression of Change plus Caregiver Input (CIBIC-plus) measured at weeks 8, 16, and 24 and the additional cognition composite measured at the baseline and endpoint.

The CIBIC-plus is a global assessment of change based on a comprehensive, semi-structured interview that includes caregiver supplied information. It is a 7-point rating scale ranging from 1 to 7, where 1 represents markedly improved; 4 represents no change; and 7 represents markedly worse

The additional cognition composite was calculated by the average of the T scores of speed of processing (Category Fluency), working memory (Wechsler Memory Scale-Third Edition, Spatial Span), and verbal learning and memory tests (Wechsler Memory Scale-Third Edition, Word Listing). The raw score of speed of processing, working memory, and verbal learning and memory tests was standardized to a T score with a mean of 50 and an SD of 10 for making each test comparative. The additional cognition composite was applied in combination with ADAS-cog to make the cognitive assessment more thorough. Decrease in processing speed has been found to be associated with aging. Working memory and verbal learning/memory also decline in patients with AD.

Systemic side effects of treatments were evaluated by means of physical and neurological examinations and laboratory tests including CBC and biochemistry and reviewed by applying the Udvalg for Kliniske Undersogelser Side-effects Rating Scale at the baseline and weeks 8, 16, and 24.

Clinical ratings were performed by the research psychiatrists and neurologists who were trained and experienced in the rating scales. Inter-rater reliability was analyzed with the analysis of variance test. Only raters reaching the intra-class correlation coefficients of ≧.90 during prestudy training were allowed to rate the study patients. To maintain high inter-rater reliability and to prevent rater drift, raters met at least once/quarter for training and reliability retesting. To minimize inter-rater variability, each individual patient was assessed by the same research psychiatrist or neurologist throughout the trial.

### Data Analysis

Chi-square test (or Fisher's exact test) was used to compare differences of categorical variables and Student two-sample t test (or Mann-Whitney U test if the distribution was not normal) was used for continuous variables between two treatment groups. Mean changes from the baseline in repeated measure assessments (ADAS-cog) were assessed with the generalized estimating equation (GEE) method with treatment, visit, and treatment-visit interaction as fixed effects and intercept as the only random effect and the baseline value as the covariance. The GEE analyses were performed with the SAS/STAT (SAS Institute, Cary, N.C.) “PROC GENMOD” procedure with first-order autoregressive working correlation structure with the marginal model instead of the mixed effect model. Therapeutic effect sizes (Cohen's d) were used to determine the magnitude of improvement for the continuous variables resulting from sodium benzoate treatment compared with the placebo.

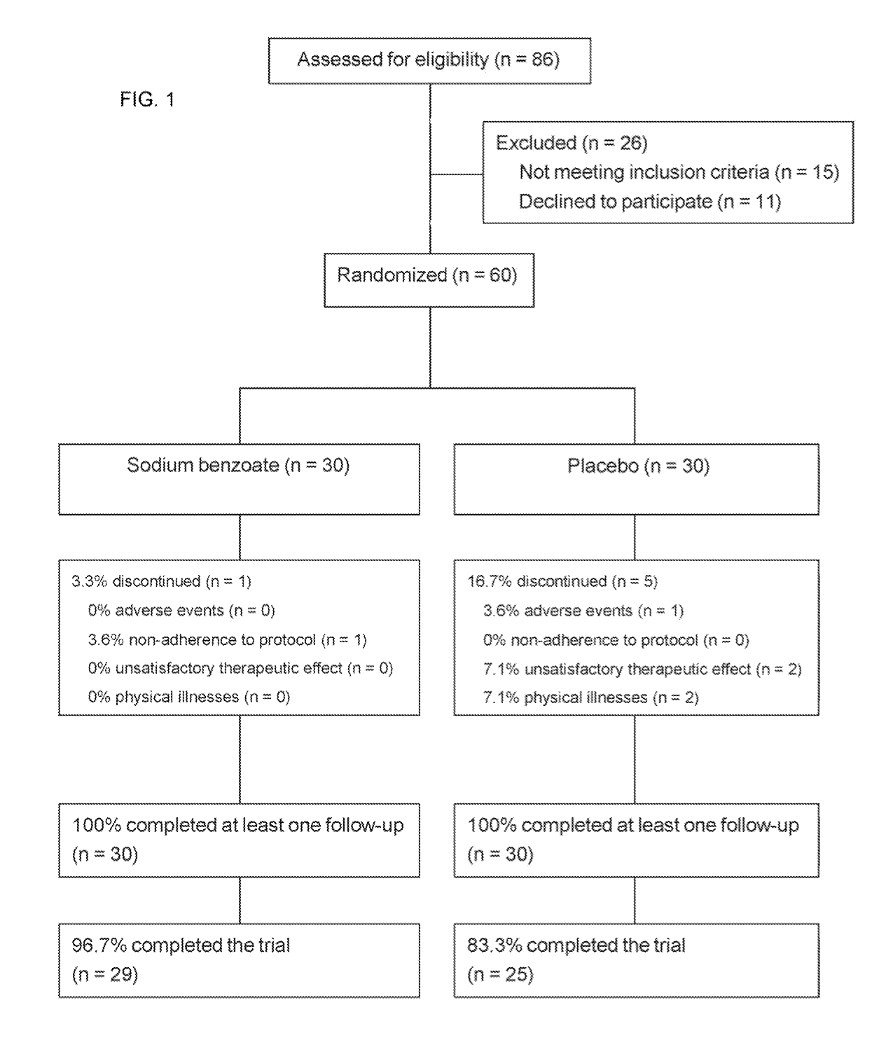

Finally, all of the 60 randomized patients completed at least one follow-up, and 50 (90%) of them completed the 24-week trial (FIG. 1). No imputation for the incomplete data was used for the GEE analysis.

There were no baseline scores for the CIBIC-plus, because this is scored as a judgment of change from the baseline. Differences in CIBIC-plus scores at weeks 8, 16, and 24 and endpoint between groups were assessed by Student two-sample test (or Mann-Whitney U test if the distribution was not normal).

Fisher's exact test was used to compare differences in the dropout rates between the two groups. Cohen's w was applied for determining the effect size of categorical variables. All data were analyzed by IBM SPSS Statistics (version 18.0; SPSS, Chicago, Ill.) or SAS version 93. All p values for clinical measures were based on two-tailed tests with a significant level of .05.

### Results

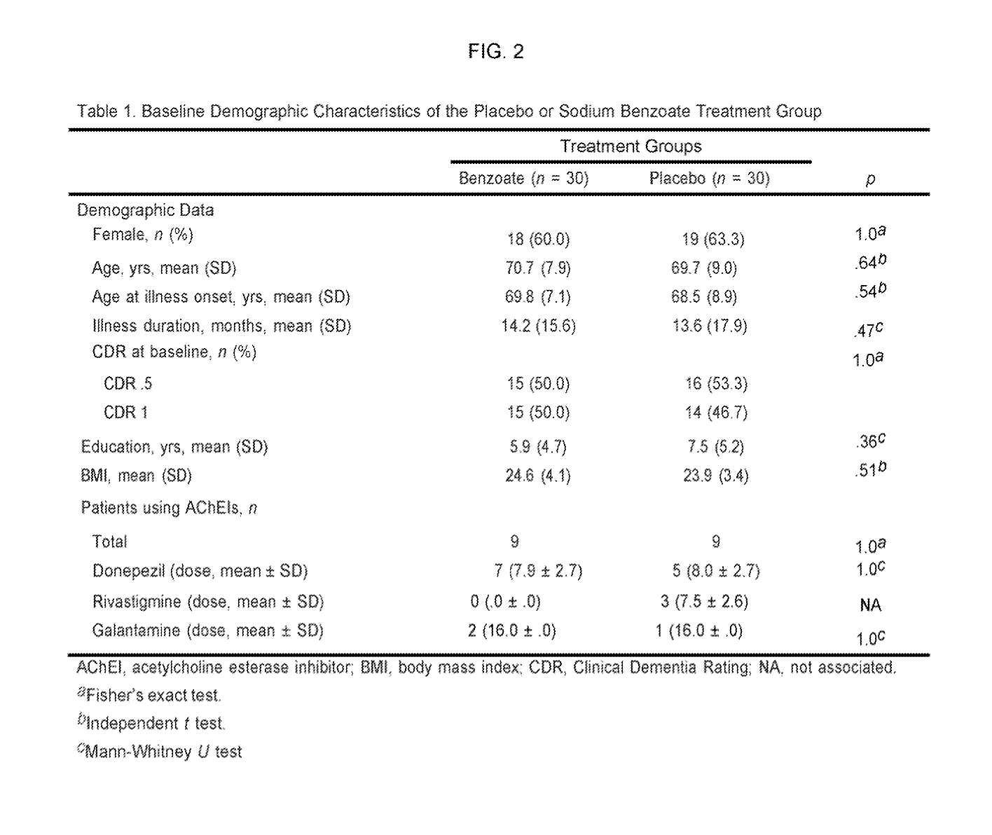

Sixty patients were eligible and randomized (FIG. 1). Demographic data, education level, age at illness onset, illness duration, CDR, body mass index, and AChEI use at baseline were similar between the benzoate group (n=30) and the placebo group (n=30)(p>.05) (FIG. 2, Table 1). The AChEI doses were within the therapeutic range and similar between two groups (FIG. 2, Table 1). Mean dose of sodium benzoate at weeks 8, 16, and 24 were 275.0 76.3 mg/day, 525.0±100.6 mg/day, and 716.7 182.6 mg/day, respectively. FIG. 2 is a table which shows the baseline demographic characteristics of placebo or sodium benzoate treatment.

### Outcome Measures

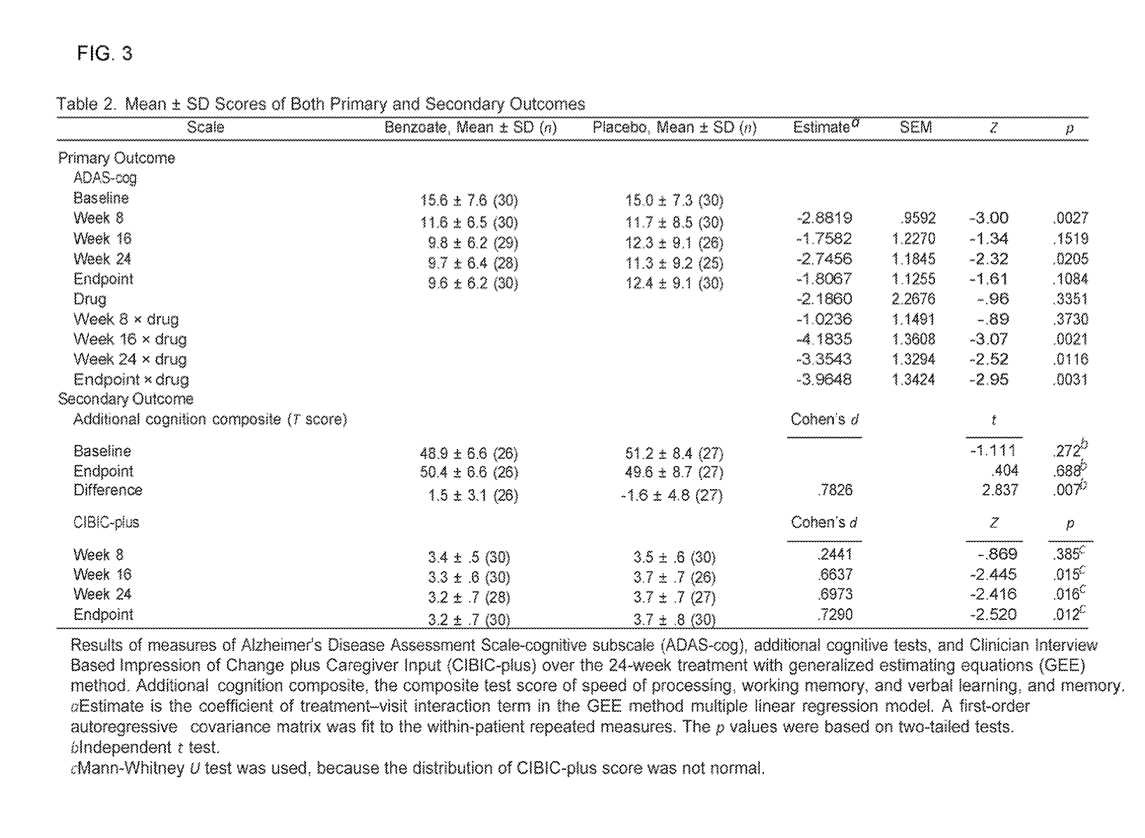

The mean ±scores of both primary and secondary outcomes—including ADAS-cog, additional cognition composite, and CIBIC-plus—of the two groups of patients are shown in FIG. 3, Table 2. At week 0 (baseline), there were no significant differences between the two groups in ADAS-cog and additional cognition composite (p=.75 and p=.27, respectively)

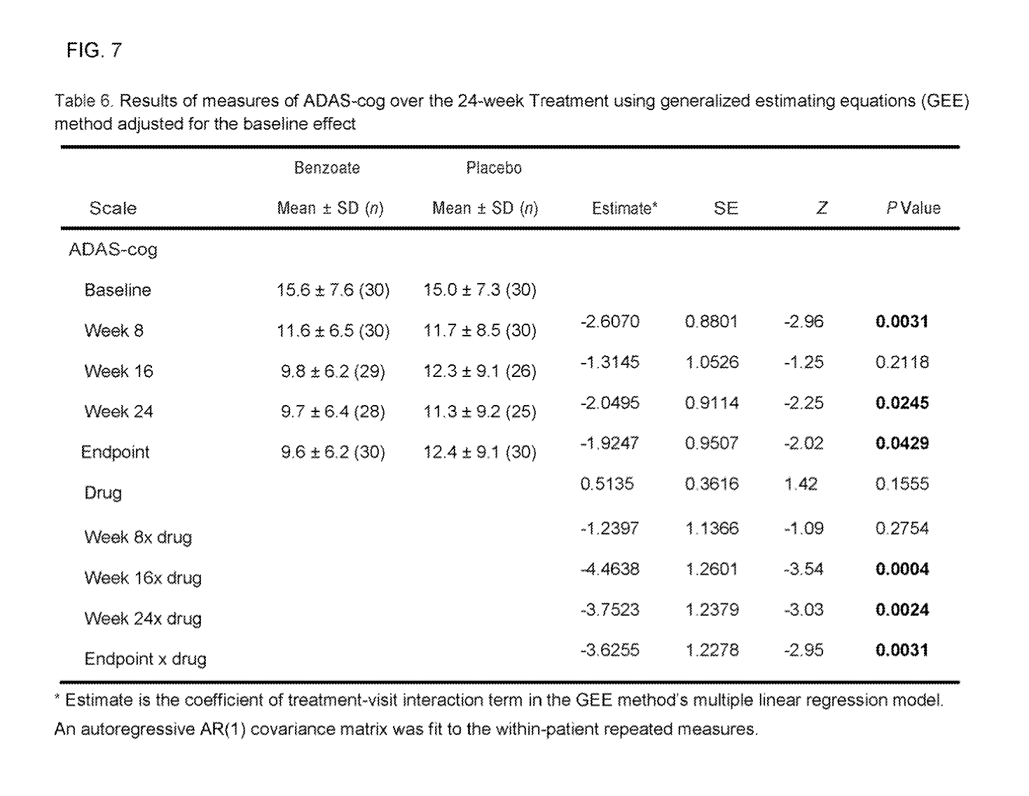

For the primary outcome, sodium benzoate produced greater improvement in ADAS-cog score than the placebo therapy throughout the study (mean differences from baseline were 3.8, 5.4, 5.9, and 5.9 in the benzoate group and 2.4, 1.7, 2.7, and 1.7 in the placebo group, at weeks 8, 16, 24, and endpoint; p=.3730,p=.0021, p=.0116, and p=.0031, respectively), with effect size of .86 at the end of the study (FIG. 3, Table 2). The results were similar when the baseline ADAS-cog score was controlled in the GEE model (FIG. 7, Table 6).

For the secondary outcomes, sodium benzoate was better than placebo in the additional cognition composite at endpoint (p=.007, effect size =.78), Benzoate treatment also produced greater improvement in CIBIC-plus score than placebo therapy at week 16 (p=.015). week 24 (p=.016), and endpoint (p=.012, effect size =.73 at endpoint) (FIG. 3, Table 2).

The dropout rate (3.3%) of the sodium benzoate group tended to be lower than that (16 7%) of the placebo group, yet insignificantly (p=.195).

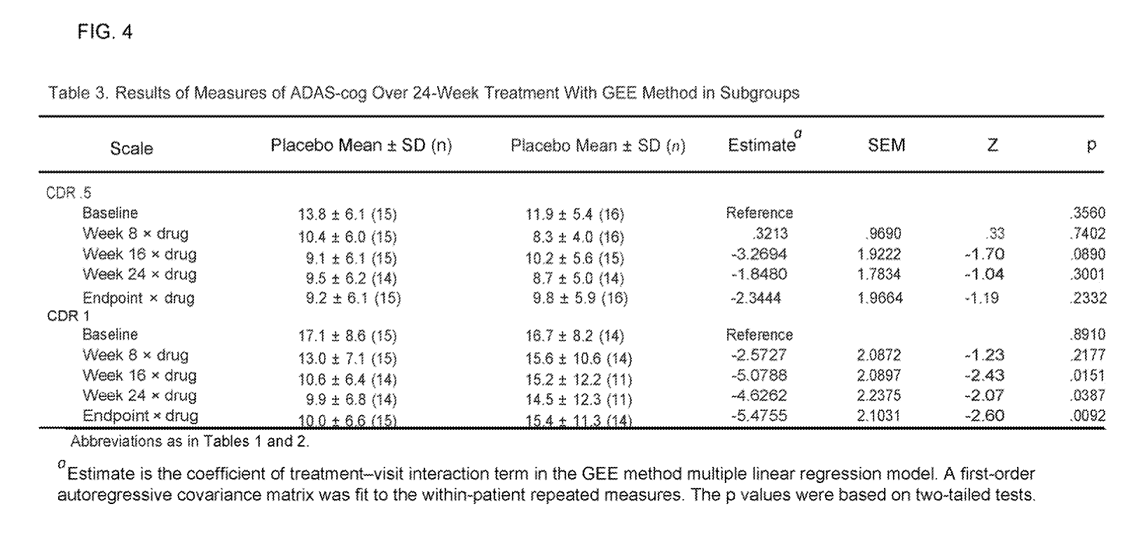

For subgroup analysis, we further examined efficacy of sodium benzoate versus placebo in CDR .5 and CDR 1 subgroups. For ADAS-cog, sodium benzoate produced greater improvement than placebo therapy at weeks 16 and 24 and endpoint (p=.0151, p=.0387, and p=.0092, respectively) in the CDR 1 subgroup. However, sodium benzoate was not superior to the placebo therapy in the CDR .5 subgroup throughout the study (p>.05) (FIG. 4, Table 3).

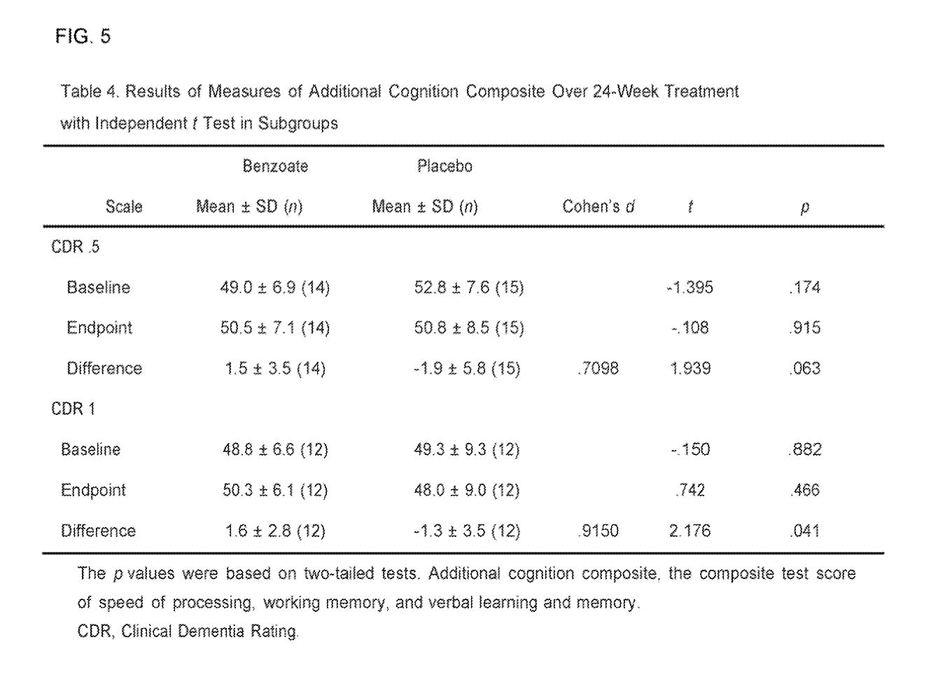

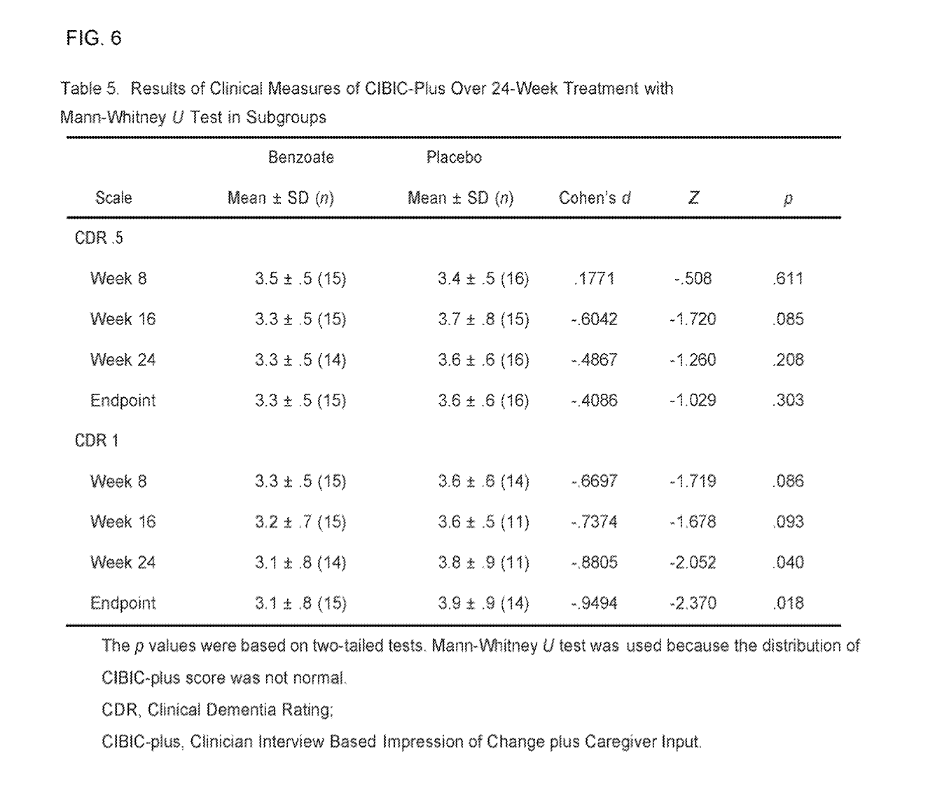

Sodium benzoate showed better efficacy in the CDR 1 subgroup (p=.041) and borderline significance in the CDR .5 subgroup (p=.063) in improving the additional cognition composite (FIG. 5, Table 4), For CIBIC-plus, sodium benzoate produced greater improvement than placebo therapy at week 24 and endpoint (p=.040 and p=.018, respectively) in the CDR 1 subgroup but not in the CDR .5 subgroup (FIG. 6, Table 5).

### Adverse Effects

Both sodium benzoate and placebo were well-tolerated. Only one patient in the placebo group reported dizziness at week 16. The side effect was mild and did not warrant medical treatment. There was no reported side effect in the sodium benzoate group assessed by the Udvalg for Kliniske Undersogelser Side-effects Rating Scale at all visits. No dropout was due to side effect.

The routine blood cell count and chemistry were all within the normal ranges and remained unchanged after treatment (data not shown).

It is critical to identify and treat AD as early as possible, potentially to arrest its progression. The current study is the first to apply a DAAO inhibitor, sodium benzoate herein, as a novel treatment for the early stage of cognitive decline. The result showed that sodium benzoate had better efficacy than placebo in improving ADAS-cog score, additional cognition composite (consisting of speed of processing, working memory, and verbal learning and memory), and global function in all subjects as a whole. Subgroup comparisons found that benzoate was beneficial for all outcome measures among patients with mild AD. In the aMCI subgroup, sodium benzoate showed borderline significance in improving the cognition composite, but not in ADAS-cog. This is probably due to the small sample size lacking the power to detect a smaller effect than mild AD. Moreover, sodium benzoate also demonstrated favorable safety profiles.

Although NMDAR activity is essential for cognitive function, its role in AD is still not fully understood. The NMDAR overactivation by glutamate results in cell death. The excitotoxicity is one of the theories of AD, particularly in the late stage. Memantine, a low-affinity, voltage-dependent uncompetitive NMDA antagonist, has been used for the treatment of moderate-severe AD. The current study suggests that NMDAR enhancement is beneficial for early and mild dementia. There is an age-related decrease of glutamate content and synthesis in human cerebral cortex and hippocampus, of which the most significant and consistent finding is decreased density of NMDAR in elderly persons and in patients with AD. Lower levels of D-serine and higher levels of L-serine in the serum were also observed in patients with AD. Therefore, in addition to the cholinergic system, dysfunction of NMDA neurotransmission might also play an important role in the pathophysiology of AD.

With regard to the dosing strategy, sodium benzoate provided better efficacy than placebo at week 16 and week 24, with the mean dose of 525 mg/day and 716 mg/day respectively, possibly implying that sodium benzoate at 500-750 mg/day is more effective than 250 mg/day. Another possibility is that longer sodium benzoate treatment duration yields better treatment response. Further studies COM paring different doses of sodium benzoate with a fixed-dose design are required for finding the time to response and the optimal dose for the treatment of mild AD or MCI. The AChEIs are commonly used for the treatment of AD but not recommended for the treatment of MCI due to weak beneficial effects and risk of side effects. The consensus statement from the British Association for Psychopharmacology concludes that neither AChEIs nor memantine is effective in treating MCI. Other compound commonly used for the treatment of MCI, such as vitamin E, folic acid, omega-3 fatty acid, piracetam, and ginkgo biloba, also failed to show convincing evidence for a cognitive enhancing effect. Sodium benzoate is generally safe; however, its efficacy for aMC did not reach statistical significance in the current small-sized study either, although it suggested a trend of improvement.

Although AD AS-cog is widely used in AD clinical trials, it might be less sensitive for MCI. One of the strategies to improve the detection of responsiveness for MCI is to add additional cognitive tests. People with MCI have been found to be impaired in neuropsychological functions such as speed of processing, working memory, and verbal learning and memory. In the aMCI subgroup of the present study, sodium benzoate showed borderline significance in improving the additional cognition composite, consisting of speed of processing, working memory, and verbal learning/memory, but not in ADAS-cog score. Our result echoes the suggestion that additional neuropsychological tests that are more sensitive to subtle deficits should also he applied in the trials for MCI.

In addition, sodium benzoate also did not improve CIBIC-plus score in the aMCI subgroup. A possible explanation is a ceiling effect that functional impairment is minimal in the MCI individuals, thereby restricting the space for further improvement More sensitive and specific measurements for the function of MCI individuals, such as Clinical Dementia Rating Sum of Boxes or Alzheimer's disease Cooperative Study scale for ADL in MCI, can be applied in the future studies for MCI.

This study is limited by its small sample size, which led to underpowered results particularly in the subgroup analysis of aMCI and a lack of MCI-specific functional assessments. We have found that benzoate can increase the brain mass by magnetic resonance imaging study.

Very high levels of DAAO are detected in the cerebellum of adult brain, whereas the activity of DAAO is low in the forebrain, such as prefrontal cortex and hippocampus, despite robust expression. The cellular localization and function of DAAO are likely different between forebrain and cerebellum: it is glial in the cerebellum but mainly neuronal in the cerebral cortex. However, the effect of DAAO inhibitors on forebrain D-serine level is inconsistent. Most DAAO inhibitors can cause a measurable increase in D-serine in the forebrain as observed in the cerebellum, whereas some inhibitors might not. Nevertheless, cerebellum is involved in cognition. Sodium benzoate might exert its procognitive effects by not only cerebral but also cerebellar mechanism.

Despite the aforementioned limitations, this study suggests that sodium benzoate, a DAAO inhibitor, is beneficial for cognitive and overall function in patients with early-phase AD.

The foregoing description of the exemplary embodiments of the invention has been presented only for the purposes of illustration and description and is not intended to be exhaustive or to limit the invention to the precise forms disclosed. Many modifications and variations are possible in light of the above teaching.

The embodiments and examples were chosen and described in order to explain the principles of the invention and their practical application so as to enable others skilled in the art to utilize the invention and various embodiments and with various modifications as are suited to the particular use contemplated. Alternative embodiments will become apparent to those skilled in the art to which the present invention pertains without departing from its spirit and scope. Accordingly, the scope of the present invention is defined by the appended claims rather than the foregoing description and the exemplary embodiments described therein.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

What is claimed is:

1. A method of treating dementia, comprising:
administering to a subject afflicted with dementia a composition comprising:
(a) an effective amount of benzoic acid, a salt, an ester or a derivative thereof; and
(b) a pharmaceutically acceptable carrier or vehicle,
wherein the subject is optionally concurrently administered an acetylcholine esterase inhibitor but not administered any other neuropharmaceutical.

2. The method of claim 1, wherein the administering step is performed daily for a period selected from the group consisting of more than 6 weeks, at least 8 weeks, at least 16 weeks, at least 20 weeks, and at least 24 weeks.

3. The method of claim 1, wherein the salt thereof is sodium benzoate.

4. The method of claim 3, wherein the amount of sodium benzoate administered is at least 250 mg per daily dosage unit.

5. The method of claim 4, wherein the amount of sodium benzoate administered is at least 500 mg per daily dosage unit.

6. The method of claim 1, wherein the amount of the benzoic acid, salt, ester, or derivative thereof administered is at least 200-2000 mg per daily dosage unit.

7. The method of claim 6, wherein the salt thereof is sodium benzoate and the amount administered is at least 250-750 mg per daily dosage unit.

8. The method of claim 1, wherein the administering step comprises:
(a) administering to the subject sodium benzoate about 200-500 mg per daily dosage unit for the first treatment period;
(b) administering to the subject sodium benzoate about 400-1000 per daily dosage unit for the second treatment period; and
(c) administering to the subject sodium benzoate about 500-1000 per daily dosage unit for the second treatment period.

9. The method of claim 1, wherein the subject is afflicted with Alzheimer disease and/or mild cognitive impairment.

10. The method of claim 1, wherein the subject is afflicted with an early phase Alzheimer disease and/or amnestic mild cognitive impairment.

11. The method of claim 1, further comprising:
measuring and assessing the cognitive function of the subject.

12. The method of claim 11, wherein the measuring step is performed by using an Alzheimer's Disease Assessment Scale-cognitive subscale instrument.

13. The method of claim 1, wherein the subject is not concurrently administered an acetylcholine esterase inhibitor or any other neuropharmaceutical.

14. The method of claim 11, wherein the measuring step measures the scores of speed of processing, working memory, and verbal learning and/or memory tests.

15. The method of claim 11, wherein the measuring step measures Clinician's interview-Based Impression of Change plus Caregiver input.

16. A method of treating dementia, comprising:
administering to a human subject in need of treating the dementia, a composition consisting essentially of:
(a) benzoic acid, a salt, an ester, or a derivative thereof in an amount effective for inhibition of D-amino acid oxidase in the brain of the subject; and
(b) a pharmaceutically acceptable carrier or vehicle,
wherein the subject is optionally concurrently administered an acetylcholine esterase inhibitor but not administered any other neuropharmaceutical.

17. The method of claim 16, wherein the human subject is not concurrently administered any acetylcholine esterase inhibitor or any other anti dementia compound.

18. A method of treating dementia in a human subject, comprising administering to the human subject a composition comprising:
(a) an effective amount of a D-amino acid oxidase inhibitor; and
(b) a pharmaceutically acceptable carrier or vehicle,
wherein the subject is not administered any other neuropharmaceutical.

19. The method of claim 18, wherein the D-amino acid oxidase inhibitor is at least one selected from the group consisting of benzoic acid, a salt, an ester, and a derivative thereof.

20. The method of claim 19, wherein the D-amino acid oxidase inhibitor is sodium benzoate.
